(19) United States
(12) Patent Application Publication
(10) Pub. No.: US 2009/0017111 A1
(43) Pub. Date: Jan. 15, 2009

(54) TOLTERODINE BEAD

(76) Inventor: Dennie J. M. van den Heuvel, Beuningen (NL)

Correspondence Address: SYNTHON IP INC, 7130 HERITAGE VILLAGE PLAZA, STE 202, GAINESVILLE, VA, 20155, US

(21) Appl. No.: 12/167,384

(22) Filed: Jul. 3, 2008

Related U.S. Application Data
(60) Provisional application No. 60/947,833, filed on Jul. 3, 2007.

(51) Int. Cl. A61K 9/48; A61K 9/14; A61P 13/02; A61K 31/135

(52) U.S. Cl. 424/452; 424/494; 514/648

(57) ABSTRACT

A controlled release tolterodine bead is formed having a microcrystalline cellulose core, a PVP-containing water soluble coating, a tolterodine drug layer, and a controlled release layer.

The present application claims the benefit of priority under 35 U.S.C. § 119(e) from U.S. Provisional Application Ser. No. 60/947,833, filed Jun. 3, 2007, the entire contents of which are incorporated herein by reference.

### BACKGROUND OF THE INVENTION

The present invention relates to a controlled release bead comprising tolterodine, a process for preparing it, its use for the manufacturing of a pharmaceutical dosage form, a pharmaceutical dosage form comprising it, and the use of the pharmaceutical dosage form.

### DESCRIPTION OF THE RELATED ART

Pharmaceutical formulations based on controlled release beads comprising an inert core, such as a sugar sphere, coated with a drug-containing layer and an outer membrane layer for controlling the release rate of the drug are well known in the art. An example of such a controlled release bead can be found in WO96/01621 and its equivalent U.S. Pat. No. 5,783,215. The specifications of these two patents indicate that the presence in certain amounts of a hydrophilic polymer in the drug layer can provide advantageous mechanical properties and may, for the specific drug furosemid, provide for favorable control over dissolution properties below pH 4.

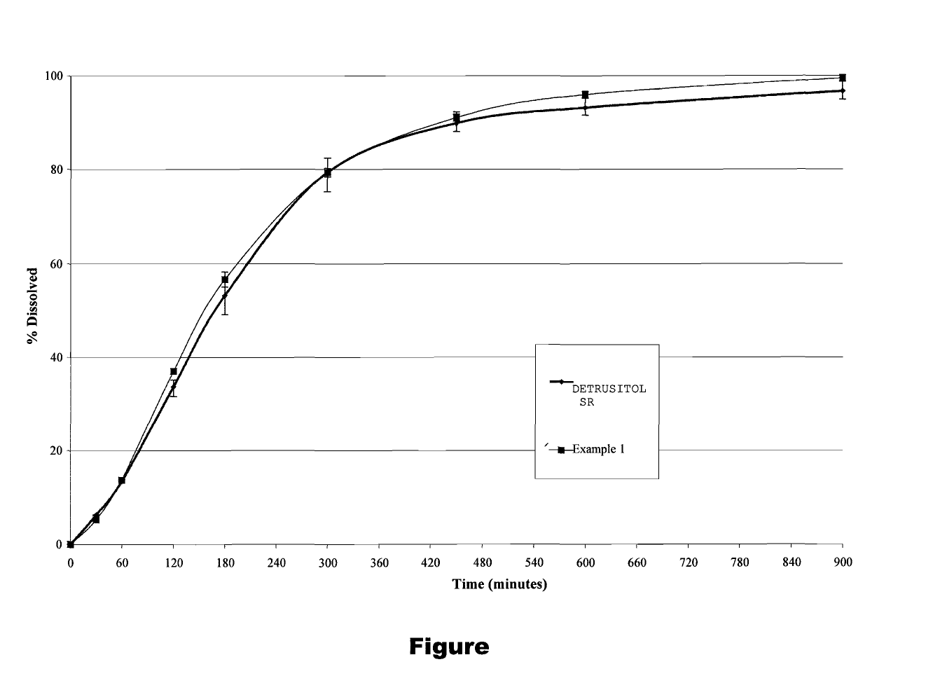

WO00/27364 and corresponding U.S. Pat. No. 6,911,217 relate to controlled release beads containing (i) an inert core, (ii) a water-insoluble polymer layer surrounding the core (iii) a drug layer thereon, and (iv) a controlled release polymer layer. The specification teaches that it was previously “not uncommon” to apply a water-soluble polymer layer between the core and the drug layer, known as a “sealcoat,” to the beads as described in U.S. Pat. No. 5,783,215. Such a water-soluble sealcoat would be present in a small amount, e.g. 1-3%. According to WO00/27364 and U.S. Pat. No. 6,911,217, the purpose of the sealcoat was to isolate the drug from the core surface to prevent any possible chemical interaction and/or to provide a smooth surface on the core with more consistent surface area to thereby obtain improved coating quality and reduced lot-to-lot variations. In contrast, the purported invention in WO00/27364 and U.S. Pat. No. 6,911,217 relates to the use of a sealcoat made of a water-insoluble material that serves to enhance the drug release profile. As seen in FIG. 1 of these patents, the in vitro release of tolterodine becomes slower and more zero-order as the amount of the water-insoluble sealcoat increases from 0% to 14%.

Pharmacia & Upjohn, now Pfizer Inc., markets a prolonged-release capsule formulation of tolterodine tartrate under such brand names as DETROL LA™ in the U.S. and DETRUSITOL SR™ in Europe. Tolterodine is a well known pharmaceutical substance and is useful for treatment of urinary disorders such as overactive bladder. Its chemical name is (R)-N,N-disopropyl-3-(2-hydroxy-5-methylphenyl)-3-phenylpropanamine and it has been disclosed in U.S. Pat. No. 5,382,600. In pharmaceutical applications, tolterodine is typically used in the form of a salt with L-tartaric acid, commonly referred to as tolterodine tartrate. According to the FDA Orange Book, the commercial product sold in the U.S. is covered by U.S. Pat. No. 6,911,217, discussed above. According to this patent, the bead composition is preferably adjusted so that the in vitro dissolution of tolterodine is not more than 30% after 1 hour, from 40 to 85% after 3 hours and not less than 80% after 7 hours.

U.S. Pat. No. 6,630,162 and U.S. Pat. No. 6,770,295 also relate to tolterodine-containing beads and capsule formulations. These patents describe controlled-release formulations, similar to those in U.S. Pat. No. 6,911,217, that obtain certain release profiles or blood plasma levels, respectively.

In each of the examples of the invention, the water-insoluble polymer ethylcellulose, under the brand name SURELEASE® (Colorcon, Inc. West Point, Pa., U.S.A.), is used as a sealcoat. The same brand name polymer is also used as the sealcoat in the examples of U.S. Pat. No. 6,911,217.

WO 2004-105735 teaches that it is possible to obtain the suitable release profile of tolterodine from coated pellets without the use of the intermediary coating (sealcoat). That is, the tolterodine-containing layer coat is directly coated on the inner core. This possibility was also studied in the U.S. Pat. No. 6,911,217, however it was shown that after two hours the release rate failed to maintain the desired zero order release rate and released the tolterodine too quickly.

Recently an application was published, WO 2007/029087, which is directed to improved cores for controlled release formulations. It describes a bead which has an inert core comprising ethyl cellulose and optionally one or more water soluble or swellable excipients, a first layer comprising the active ingredient and a hydrophilic polymer, and a second layer comprising a polymer which is effective for controlling the release of the active ingredient, wherein tolterodine is specifically mentioned as the active ingredient.

It would be desirable to provide an alternative composition of pharmaceutical pellets comprising tolterodine that provided controlled release of the active. In particular, an alternative bead formulation that allows for good scale up and commercial manufacture and which preferably provides a release rate profile similar or equivalent to the marketed controlled release capsules.

### SUMMARY OF THE INVENTION

The present invention relates to a controlled release bead comprising tolterodine or an acid addition salt thereof as the active substance.

A first aspect of the invention is a controlled release bead comprising
i) a microcrystalline cellulose core unit having a diameter of 100-2000 micron which generally constitutes 50-90 wt % of the total weight of the bead composition,
ii) a water soluble coat surrounding said core unit and comprising a vinyl pyrrolidone polymer, which coat generally constitutes 2-6 wt % of the total weight of the bead composition,
iii) a drug layer comprising tolterodine or a pharmaceutically acceptable salt thereof and a pharmaceutically acceptable binder, which layer generally constitutes 2-6 wt % of the total weight of the bead composition, and
iv) a controlled release layer comprising a pH independent polymer, which is a polyacrylate polymer, which polymer generally constitutes 2-17 wt % of the total weight of the bead composition.

A second aspect of the invention is a process for preparing a controlled release bead of the invention comprising the steps
a) providing a core unit with a diameter size of 100-2000 micron,
b) applying a water soluble coat comprising a vinyl pyrrolidone polymer on said core unit,
c) applying the drug layer comprising tolterodine or a pharmaceutically acceptable salt thereof and a pharmaceutically acceptable binder on the water soluble coat, and
d) applying a controlled release layer comprising a pH independent polyacrylate polymer on the drug layer.

A third aspect of the invention is a pharmaceutical dosage form comprising a plurality of the above-mentioned beads.

A fourth aspect of the invention is pharmaceutical dosage form according to the invention for the treatment of urinary and gastrointestinal disorders.

A fifth aspect of the invention is the use of a plurality of beads according to the invention for the manufacturing of a pharmaceutical dosage form.

A sixth aspect of the invention is the use of a copolymer of vinyl pyrrolidone and vinyl acetate in a ratio of 6 to 4 on a microcrystalline cellulose core as a platform for coating a drug layer and controlled release layer thereover.

### DESCRIPTION OF THE FIGURES

The FIGURE shows a dissolution profile of a capsule comprising a plurality of the beads of Example 1 and of commercial capsules of Detrusitol SR™ (“commercial product”). The release of tolterodine (shown as % TTD) was measured at 37° C. in 800 ml of USP phosphate buffer pH 6.8 on a USP dissolution test apparatus 1 at 100 rpm. The data in the FIGURE were obtained by an HPLC method and are uncorrected.

### DETAILED DESCRIPTION OF THE INVENTION

The present invention relates to a controlled release bead comprising tolterodine. Tolterodine within the present invention shall mean tolterodine or a pharmaceutically acceptable salt thereof, preferably tolterodine tartrate if not indicated specifically to the contrary. Percentages within the invention shall mean weight percentages (wt %) unless otherwise stated.

The controlled release beads of the invention are comprised of a microcrystalline cellulose core unit, a water soluble coat surrounding said core unit, a drug layer comprising tolterodine and a pharmaceutically acceptable binder and a controlled release layer comprising a pH independent polymer or copolymer. The “pH independent” in this context means that the permeability, and accordingly the release characteristics, of the controlled release polymer or copolymer layer is not substantially influenced by the pH. Further layers may additionally be present, although typically either no additional layers are present or an optional outermost film coat layer is additionally present if desired. Further details of the bead components are set forth below.

The microcrystalline cellulose core unit of the beads is any core or seed that contains microcrystalline cellulose and is typically a commercially available microcrystalline cellulose sphere such as Cellets™ or Celpheres™. The size of the core unit typically has a diameter within the range of 100-2000 microns, preferably the diameter of the core unit is within the range of 710-1000 microns. Generally the core unit constitutes 50-90 wt % of the total weight of the bead composition. In some embodiments, the core unit is 70-90 wt %, while in other embodiments it is preferably 60-70 wt % of the total weight of the bead composition. One benefit of using a microcrystalline cellulose unit core is that coating thereof is relatively easy such that hardly any erosion or dissolving of the core unit occurs during coating. Due to this, the coating rate may be increased (e.g., in comparison with known sugar cores) which reduces the process time and therewith the production costs without adversely affecting the quality of the beads. Also the thickness of the coat layers on the bead may be better controlled. The calculated yields of the process are more reliable since hardly any core material is lost during the different coating processes.

The cellulose core unit is primarily surrounded by a water soluble coat which contains a vinyl pyrrolidone polymer. A vinyl pyrrolidone polymer in this context includes both homopolymers and copolymers thereof, the latter generally containing at least 20% by mol of the vinyl pyrrolidone moiety in relation to other co-monomers. Preferably a copolymer of vinyl pyrrolidone and vinyl acetate is used. Such a copolymer typically has a molar ratio of about 6:4, respectively (e.g., Kollidon VA64 or more generically “PVP VA64”). The use of such a copolymer as the water soluble layer on a microcrystalline cellulose sphere is a specific aspect of the invention and provides a useful basis for making the tolterodine beads of the present invention as well as for supporting other drug layers instead of tolterodine. Other polymers can be present in the water soluble coat as well, though typically no other polymers are present and the water soluble coat is comprised mainly of the vinyl pyrrolidone polymer, e.g. at least 70%, typically at least 90%. The choice of polymer(s) used in the water soluble coat may have an influence on the lag time during dissolution of the bead, which is preferably a relatively short lag time. If an increased lag time is needed, however, HPMC may be used including to the exclusion of the vinyl pyrrolidone polymer, though this is seldom desired. Typically the water soluble coat surrounding the core constitutes 2-6 wt % of the total weight of the bead composition.

The drug layer comprises tolterodine, preferably tolterodine tartrate or another water soluble pharmaceutically acceptable salt of tolterodine, together with a pharmaceutically acceptable binder. Typically the binder is a hydrophilic polymer. Convenient hydrophilic polymer binders include hydroxypropyl methyl cellulose (HPMC). Among the commercially available grades, a low viscosity HPMC is generally desired such as Methocel E5™. However, practically any hydrophilic polymer having a sufficient binding property such as PVP, starch, hydrophilic cellulose derivatives, hydrophilic acrylate or methacrylate polymers may be used. The drug layer typically constitutes 2-6 wt % of the total weight of the bead composition. Tolterodine generally constitutes 40-70 wt % of the total weight of the drug layer.

The outer controlled release layer comprises a pH independent polymer, especially a polyacrylate polymer. The term “pH independent” was explained above. The term “polyacrylate polymer” is intended to be used in its broadest sense and includes polyacrylates and polymethacrylates as well as copolymers thereof. The polyacrylate polymer may be nonionic (neutral) or ionic. Typically a nonionic polyacrylate polymer used in the controlled release layer generally has an average molecular weight of 400,000 to 1,200,000, and more typically 500,000 to 900,000. A particularly useful nonionic polyacrylate polymer is a copolymer of ethyl acrylate and methyl methacrylate, such as found in the commercially available Eudragit NM30D™. Eudragit NM30D™ is obtainable as a ready to use aqueous dispersion containing about 30% of solid material and 0.7% Macrogol Stearyl ether as an emulsifier. The copolymer has an average molecular weight of about 600,000. An ionic polyacrylate polymer useful in the controlled release layer typically has an average molecular weight of 100,000 to 200,000. A particularly useful ionic polyacrylate polymer is a copolymer of ethyl acrylate, methyl methacrylate and trimethylammonioethyl methacrylate chloride, such as found in the commercially available Eudragit RL and/or Eudragit RS. Eudragit RL and Eudragit RS are obtainable as a ready to use solution or as a solvent-free powder, respectively. A combination of two or more brands or types of the above polymers is also useful.

The amount of polyacrylate polymer in the controlled release layer is typically at least 30% and generally at least 50% based on the total weight of the controlled release layer. Typically, this polymer constitutes 2-17 wt % of the total weight of the bead composition. In some embodiments it is advantageous to further include an anti-tacking agent such as talc (which typically constitutes about 10-60 wt %, preferably 25-50%, of the total weight of the layer) and/or glyceryl monostearate (which typically constitutes about 1-5 wt %, preferably 2.5-5%, of the total weight of the layer). Silicon dioxide, such as the SYLOID brand from W.R. Grace, can also be included in the layer. The bead of the invention releases the active ingredient preferably in a diffusion controlled manner combined with the swelling capacity of the core material. Alternatively, though it is not generally necessary or desired, the controlled release layer may be combined with a pore forming agent such as HPMC or a plasticizer such as triacetin or a polysorbate to obtain a suitable release profile.

Optionally the bead of the invention may also comprise an outermost film coat for improving the mechanical properties of the bead. Preferably such an outermost film coat is not a functional coat, i.e., it does not substantially modify the controlled release rate of the bead. Such an outermost film coat comprises preferably hydroxypropyl methyl cellulose and/or talc and constitutes about 0.5-2 wt % of the total weight of the bead composition, if present.

The beads of the present invention are controlled release beads, meaning in a broad sense that immediate release of the tolterodine has been disrupted. However, preferred embodiments of the invention exhibit the following release rate of tolterodine in a phosphate buffer pH 6.8: not more than 30% (preferably 1-25%) at 1 hour, from 35 to 85% (preferably 40-65%) at 3 hours and not less than 80% at 7 hours. In particular, it is desirable and commercially advantageous to have a dissolution profile that would provide a product bioequivalent to the current controlled release capsules of tolterodine, e.g. DETROL LA™ and/or DETRUSITOL SR™.

The process for producing the bead of the present invention can be carried out by any conventional or suitable techniques and typically comprises the following steps:
a) providing a core unit with a diameter size specified as described above,
b) applying first a water soluble coat comprising a vinyl pyrrolidone polymer on said core unit,
c) applying secondly the drug layer comprising tolterodine or a pharmaceutically acceptable salt thereof and a pharmaceutically acceptable binder on the water soluble coat, and
d) applying thirdly a controlled release layer comprising a pH independent, preferably a polyacrylate, polymer or copolymer on the drug layer.
   Optionally a step e) of applying an outermost film coat layer is also carried out.

The coating may be performed in a fluid bed coating equipment, wherein the coats are applied stepwise on the material to be coated. The coating operations are preferably performed by spraying a solution or dispersion of the respective coating materials on the particle to be coated. The liquid carriers of the materials to be coated may be water, a pharmaceutically acceptable organic solvent, such as an aliphatic alcohol (e.g., C1-C3 alcohol), or a combination of both. Any method known in the art to apply coats on a bead may be used. After any particular coating, the coated material may be dried before applying the next coat.

After the final coating step, the beads are generally cured, usually in the same fluid bed system or in a tray drier system, by heating to a temperature of about 30-80° C. for 1-72 hours. Preferably the curing is performed at a temperature of about 35-50° C. for 2-48 hours, more typically 4-24 hours.

The bead of the invention can be formulated into a pharmaceutical dosage form. The pharmaceutical dosage form comprises a plurality of beads according to the invention. The pharmaceutical dosage form may be a capsule or a tablet. The capsule can be filled with the beads in a manner that is known in the art. Typically the capsule dosage form is filled so as to obtain 1 to 10 mg of tolterodine calculated as free base. In a specific embodiment, two or more kinds of the beads, e.g. different populations, are used as a mixture together in one capsule. The beads of one population will have a different release of tolterodine than another due to differences in the bead composition. Generally the mixture of bead populations uses beads of a faster release rate (“fast spheres”) with beads of a slower release rate (“slow spheres”) in a desired ratio, which may be from 10:90 to 90:10 wt % (fast/slow), in order to obtain an overall desired release rate. The release rate of a bead population is typically adjusted/controlled by adjusting the amount and/or composition of the controlled release layer.

The plurality of controlled release beads can also be compressed into a tablet with appropriate excipients in a manner known in the art to obtain a tablet dosage form which contains 1 to 10 mg of tolterodine calculated as free base, preferably 2 to 4 mg of tolterodine tartrate. The tablet upon dissolution disintegrates into the separate controlled release beads. Again the plurality of beads may be comprised of two or more different bead populations.

The pharmaceutical dosage forms of the invention may be used in the treatment of urinary and gastrointestinal disorders.

The following examples illustrate a bead of the invention but are not limiting the invention as such.

### EXAMPLE 1

A controlled release bead having the following manufacturing formula was prepared:

| | | |
|---|---|---|
| Core | MCC spheres | 85.60 wt % |
| Water soluble layer | PVP VA64 | 3.26 wt % |
| Drug layer | Tolterodine tartrate | 2.23 wt % |
| | HPMC (Methocel E5) | 1.80 wt % |
| Controlled release layer | Eudragit NM30D | 4.39 wt % |
| | Talc (micronised) | 2.20 wt % |
| optional layer | Talc (micronised) | 0.53 wt % |

Corresponding amounts of the ingredients were weighed in respect to 3 kg of microcrystalline cellulose spheres used as starting material. The coatings were applied by means of a fluid bed coating technology equipped with a Wurster insert.

Process

Prepare 10% PVP VA64 suspension by hydrating II 3.5 g PVP VA64 with 1027 g demiwater. Coat 3 kg MCC spheres with 1036 g PVP VA64 solution using the Glatt GPCG1 fluid bed at a product temperature of about 40° C.

Drug Layer

Prepare a suspension of Tolterodine tartrate and HPMC by hydrating 72 g of HPMC E5 in 2231 g demiwater for 24 hours. Add 89 g Tolterodine tartrate and wet with a magnetic stirrer for 15 min. Then use Ultraturax 20-30 minutes to suspend the drug in the HPMC solution. Coat 3 kg MCC spheres (comprising PVP VA64 coating) with 1994 g suspension at a product temperature of about 30° C.

CR Layer

Dilute the 153 g Eudragit NM30D (30% suspension) with 241 g demiwater and disperse 22.9 g talc for 15 minutes in the suspension. Sieve the suspension over a 250 μm screen and coat 1 kg MCC spheres (comprising PVP VA64 coating and drug layer) with 378 g Eudragit/talc suspension using the Glatt GPCG1 fluid bed at a product temperature of about 23° C.

At the end of the process, dry the spheres at 40° C. for 24 hours.

The spheres are filled into hard gelatin capsules. Filling weight is typically about 90 mg for the tolterodine tartrate 2 mg dosage form and about 180 mg for the tolterodine 4 mg dosage form.

The release rate of the product of example 1 is shown in the FIGURE along with a 3 batch average (n=18) dissolution of Detrusitol SR. The release rate was measured at 37.5° C. in 800 ml of USP phosphate buffer pH 6.8 on a USP dissolution apparatus 1 at 100 rpm. The data in the FIGURE were obtained by a HPLC method and are uncorrected.

### EXAMPLE 2

A controlled release tablet having the following manufacturing formula was prepared:

| | |
|---|---|
| Coated tolterodine spheres | 43.1 wt % |
| Lactose monohydrate | 24.4 wt % |
| MCC | 24.4 wt % |
| Sodium starch glycolate | 7.4 wt % |
| Magnesium stearate | 0.7 wt % |

The Tolterodine containing spheres of Example 1 were mixed with Lactose, MCC and sodium starch glycolate for 15 minutes in a free fall mixer at 22 rpm.

The Magnesium stearate was sieved over a 0.8 mm sieve and added to the previous blend. Mixing was continued for another 3 min at 22 rpm. Tablets were compressed with a suitable compression force on an excentric press: punch type round 8 mm, tablet weight 300 mg.

### EXAMPLE 3

A controlled release bead having the following manufacturing formula was prepared:

| | | |
|---|---|---|
| Core | MCC spheres | 77.25 wt % |
| Water soluble layer | PVP VA64 | 2.32 wt % |
| Drug layer | Tolterodine tartrate | 2.28 wt % |
| | HPMC (Methocel E5) | 1.84 wt % |
| Controlled release layer | Eudragit NM30D | 12.55 wt % |
| | Glyceryl monostearate | 0.63 wt % |
| | Polysorbate | 0.25 wt % |
| | HPMC | 1.88 wt % |
| Optional layer | HPMC | 1.00 wt % |

Corresponding amounts of the ingredients were weighed in respect to 4 kg of microcrystalline cellulose spheres used as starting material. The coatings were applied by means of a fluid bed coating technology equipped with a Wurster insert.

Process

Prepare 10% PVP VA64 suspension by hydrating PVP VA64 with demiwater. Coat 4 kg MCC spheres with 1200 g PVP VA64 solution using the Glatt GPCG1 fluid bed at a product temperature of about 40° C.

Drug Layer

Prepare a suspension of Tolterodine tartrate and HPMC by hydrating 104 g of HPMC E5 in 2145 g demiwater for 10 hours. Add 128 g Tolterodine tartrate and wet with a magnetic stirrer for 15 min. Then use Ultraturax 20-30 minutes to suspend the drug in the HPMC solution. Coat 3.75 kg MCC spheres (comprising PVP VA64 coating) with 1982 g suspension at a product temperature of about 30° C.

CR Layer

Heat up 109 g demiwater to 60-70%, add 8.2 g glycerine monostearate and mix for 10 minutes. Add 3.3 g polysorbate and 24.7 g HPMC and mix for additional 20 minutes. Add 109 g demiwater, cool down to 30° C. if needed, and mix with 550 g of the Eudragit. Coat 1 kg of MCC spheres (comprising PVP VA64 coating and drug layer) with732 g of the suspension using the Glatt GPCG1 fluid bed at a product temperature of about 23° C.

Additional Topcoat

Prepare a 10% HPMC suspension and coat the MCC spheres (comprising PVP VA64 coating, drug layer and CR layer) until a weight gain of 1-2%.

At the end of the process, dry the spheres at 40° C. for 24 hours.

The spheres are filled into hard gelatin capsules. Filling weight is typically about 90 mg for the tolterodine tartrate 2 mg dosage form and about 180 mg for the tolterodine 4 mg dosage form.

### EXAMPLE 4

A controlled release bead having the following manufacturing formula was prepared:

| | | |
|---|---|---|
| Core | MCC spheres | 68.01 wt % |
| Water soluble layer | PVP VA64 | 2.04 wt % |
| Drug layer | Tolterodine•tartrate | 2.20 wt % |
| | HPMC (Methocel E5) | 1.78 wt % |
| Controlled release layer | Eudragit RS powder | 9.99 wt % |
| | Eudragit RL powder | 1.11 wt % |
| | Talc | 11.10 wt % |
| | Triethylcitrate | 1.12 wt % |
| | HPMC | 1.67 wt % |
| Optional layer | HPMC | 1.00 wt % |

Corresponding amounts of the ingredients were weighed in respect to 4 kg of microcrystalline cellulose spheres used as starting material.

The process of making the spheres comprising the PVP layer and the drug layer is analogous to that of the Example 3.

CR Layer

Mix 148.5 g of Eudragit RS and 16.5 g of Eudragit RL with 3174 g of isopropanol and 167 g of water. Add 165 g of talc, 16.5 g of TEC and 24.7 g of HPMC to form a suspension. Coat 1 kg of the MCC spheres (comprising PVP VA64 coating and drug layer) with 3375 g of the Eudragit/talc suspension using the Glatt GPCG2 fluid bed at a product temperature of about 23° C.

Additional Topcoat

Prepare a 10% HPMC suspension and coat the MCC spheres (comprising PVP VA64 coating, drug layer and CR layer) until a weight gain of 1-2%.

At the end of the process, dry the spheres at 40° C. for 2 hours.

The spheres are filled into hard gelatin capsules. Filling weight is typically about 90 mg for the tolterodine tartrate 2 mg dosage form and about 180 mg for the tolterodine 4 mg dosage form.

### EXAMPLE 5 (“Fast Spheres”)

A controlled release bead having the following manufacturing formula was prepared:

| | | |
|---|---|---|
| Core | MCC spheres | 88.15 wt % |
| Water soluble layer | PVP VA64 | 2.64 wt % |
| Drug layer | Tolterodine tartrate | 2.60 wt % |
| | HPMC (Methocel E5) | 2.10 wt % |
| Controlled release layer | Eudragit NM30D | 2.87 wt % |
| | Glyceryl monostearate | 0.14 wt % |
| | Polysorbate | 0.06 wt % |
| | HPMC | 0.43 wt % |
| Optional layer | HPMC | 1.00 wt % |

Corresponding amounts of the ingredients were weighed in respect to 4 kg of microcrystalline cellulose spheres used as starting material. The coatings were applied by means of a fluid bed coating technology equipped with a Wurster insert.

Process

Prepare 10% PVP VA64 suspension by hydrating 130 g PVP VA64 with 1170 g demiwater. Coat 4 kg MCC spheres with 1200 g PVP VA64 solution using the Glatt GPCG1 fluid bed at a product temperature of about 40° C.

Drug Layer

Prepare a suspension of Tolterodine tartrate and HPMC by hydrating 104 g of HPMC E5 in 2145 g demiwater for 10 hours. Add 128 g Tolterodine tartrate and wet with a magnetic stirrer for 15 min. Then use Ultraturax 20-30 minutes to suspend the drug in the HPMC solution. Coat 3.75 kg MCC spheres (comprising PVP VA64 coating) with 1982 g suspension at a product temperature of about 30° C.

CR Layer

Heat up 22 g demiwater to 60-70° C. and add 1.6 g glycerine monostearate and mix for 10 minutes. Add 0.6 g polysorbate and 4.9 g HPMC and mix for 20 additional minutes. Cool suspension down to 30° C. with 22 g demiwater and mix with 110 g Eudragit. Coat 1 kg MCC spheres (comprising PVP VA64 coating and drug layer) with 146 g suspension using the Glatt GPCG1 fluid bed at a product temperature of about 23° C.

Additional Topcoat

Prepare a 10% HPMC suspension and coat the MCC spheres (comprising PVP VA64 coating, drug layer and CR layer) until a weight gain of 1-2%.

At the end of the process, dry the spheres at 40° C. for 24 hours.

The spheres are filled into hard gelatin capsules. Filling weight is typically about 90 mg for the tolterodine tartrate 2 mg dosage form and about 180 mg for the tolterodine 4 mg dosage form.

### EXAMPLE 6 (“Fast Spheres”)

A controlled release bead having the following manufacturing formula was prepared:

| | | |
|---|---|---|
| Core | MCC spheres | 85.21 wt % |
| Water soluble layer | PVP VA64 | 2.56 wt % |
| Drug layer | Tolterodine tartrate | 2.75 wt % |
| | HPMC (Methocel E5) | 2.23 wt % |
| Controlled release layer | Eudragit RS PO | 2.50 wt % |
| | Eudragit RL PO | 0.28 wt % |
| | Talc | 0.28 wt % |
| | Triethylcitrate | 2.78 wt % |
| | HPMC | 0.42 wt % |
| Optional layer | HPMC | 0.99 wt % |

Corresponding amounts of the ingredients were weighed in respect to 4 kg of microcrystalline cellulose spheres used as starting material.

The process of making the spheres comprising the PVP layer and the drug layer is analogous to that of the Example 3.

CR Layer

Mix 14.8 g Eudragit RS and 1.6 g Eudragit RS with 317 g isopropanol and 16 g demiwater. Add 1.6 g TEC and 16.5 g Talc and 2.48 g HPMC to form a suspension. Coat the 0.5 kg MCC spheres (comprising PVP VA64 coating and drug layer) with 337.5 g Eudragit/talc suspension using the Glatt GPCG2 fluid bed at a product temperature of about 23° C.

Additional Topcoat

Prepare a 10% HPMC suspension and coat the MCC spheres (comprising PVP VA64 coating, drug layer and CR layer) until a weight gain of 1-2%.

At the end of the process, dry the spheres at 40° C. for 24 hours.

The spheres are filled into hard gelatin capsules. Filling weight is typically about 90 mg for the tolterodine tartrate 2 mg dosage form and about 180 mg for the tolterodine 4 mg dosage form.

### EXAMPLE 7

A controlled release bead having the following manufacturing formula was prepared

| | | |
|---|---|---|
| core | MCC spheres | 80.06 wt % |
| Water soluble layer | PVP VA64 | 3.00 wt % |
| Drug layer | Tolterodine tartrate | 2.22 wt % |
| | HPMC (Methocel E5) | 1.80 wt % |
| Controlled release layer | Eudragit NM30D | 9.20 wt % |
| | Syloid | 3.22 wt % |
| Topcoat | Syloid | 0.50 wt % |

Corresponding amounts of the ingredients were weighed in respect to 180 kg of microcrystalline cellulose spheres used as starting material. The coatings were applied by means of a fluid bed coating technology equipped with a Wurster insert.

Process

Prepare 10% PVPVA64 suspension by hydrating 7.42 kg PVPVA64 with 66.77 kg demiwater. Coat 180 kg MCC spheres with 67.5 kg PVPVA64 solution using an Aeromatic Fielder fluid bed system at a product temperature of about 40° C.

Drug Layer

Prepare a suspension of Tolterodine tartrate and HPMC by hydrating 4.551 kg of HPMC E5 in 140.495 kg demiwater for 10 hours. Add 5.619 kg Tolterodine tartrate and use an Ultraturax for about 20 minutes to suspend the drug in the HPMC solution. Coat 175 kg MCC spheres (comprising PVPVA64 coating) with 125.6 kg suspension at a product temperature of about 30° C.

CR Layer

Sieve 67.467 kg Eudragit NM30D (30% suspension) suspension over a 200 μm screen and dilute with 109.451 kg demiwater. Disperse 7.084 kg Syloid and stir the suspension for 30 minutes. Coat 87.08 kg MCC spheres (comprising PVPVA64 coating and drug layer) with 83.6 kg Eudragit/Syloid suspension using the Aeromatic fluid bed system at a product temperature of about 24° C.

Topcoat

Disperse 1.1 kg Syloid in 9.9 kg demiwater and stir the suspension for 30 minutes. Coat 99.5 kg MCC spheres (comprising PVPVA64 coating, drug layer and Eudragit/Syloid layer) with 5 kg Syloid suspension using the Aeromatic fluid bed system at a product temperature of about 24° C.

At the end of the process, dry the spheres at 40° C. for 24 hours.

The spheres are filled into hard gelatin capsules. Filling weight is typically about 90 mg for the tolterodine tartrate 2 mg dosage form and about 180 mg for the tolterodine 4 mg dosage form.

Each of the patents and published applications mentioned above are incorporated herein by reference in their entirety. The invention having been described it will be obvious that the same may be varied in many ways and all such modifications are contemplated being within the scope of the invention as defined by the following claims.

I claim:

1. A controlled release bead comprising:
i) a microcrystalline cellulose core unit having a diameter of 100-2000 micron which constitutes 50-90 wt % of the total weight of the bead composition;
ii) a water soluble coat surrounding said core unit and comprising a vinyl pyrrolidone polymer;
iii) a drug layer comprising tolterodine or a pharmaceutically acceptable salt thereof and a pharmaceutical acceptable binder; and
iv) a controlled release layer comprising a pH independent polymer, wherein said pH independent polymer is a polyacrylate.

2. The bead according to claim 1, wherein the core unit has a diameter of 710-1000 micron.

3. The bead according to claim 1, wherein the core unit constitutes 70-90 wt % of the total weight of the bead composition.

4. The bead according to claim 1, wherein said water soluble coat surrounding said core unit constitutes 2-6 wt % of the total weight of the bead composition.

5. The bead according to claim 1, wherein said water soluble coat surrounding said core unit is a copolymer of vinyl pyrrolidone and vinyl acetate.

6. The bead according to claim 5, wherein said copolymer of vinyl pyrrolidone and vinyl acetate is in a ratio of about 6 to 4, respectively.

7. The bead according to claim 1, wherein the pH independent polymer is a copolymer of ethyl acrylate and methyl methacrylate.

8. The bead according to claim 7, wherein the pH independent polymer in the controlled release layer constitutes 2-17 wt % of the total weight of the bead composition.

9. The bead according to the claim 7, wherein the copolymer further comprises trimethylammonioethyl methacrylate chloride.

10. The bead according to claim 1, wherein said controlled release layer contains said pH independent polymer in an amount of at least 30%.

11. The bead according to claim 10, wherein said controlled release layer further comprises 10-60% of talc, 1-5 wt % of glyceryl monostearate, or both.

12. The bead according to claim 1, wherein the tolterodine is tolterodine tartrate.

13. The bead according to claim 1, wherein the pharmaceutical acceptable binder in said drug layer is hydroxypropyl methyl cellulose.

14. The bead according to claim 1, which further comprises an outermost film coat layer which constitutes 0.5-2 wt % of the total weight of the bead composition.

15. A process for preparing a bead according to claim 1, which comprises the following steps:
a) providing a microcrystalline cellulose core unit having a diameter of 100-2000 micron;
b) applying a water soluble coating that comprises a vinyl pyrrolidone polymer on said core unit;
c) applying a drug layer comprising tolterodine or its salt and a pharmaceutically acceptable binder on the water soluble coat; and
d) applying a controlled release layer comprising a pH independent polymer on the drug layer, wherein said pH independent polymer is a polyacrylate.

16. A pharmaceutical dosage form comprising a plurality of beads according to claim 1.

17. The pharmaceutical dosage form according to claim 16, wherein the dosage form is a capsule.

18. The pharmaceutical dosage form according to claim 17, wherein said capsule contains two or more populations of said beads, wherein each population has a different release rate of tolterodine from the other.

19. In a controlled release tolterodine bead comprising a core, a drug layer, and a controlled release layer, the improvement which comprises a microcrystalline cellulose core coated with a layer of a copolymer of vinyl pyrrolidone and vinylacetate in a ratio of 6 to 4, respectively.
